(19) United States
(12) Patent Application Publication
(10) Pub. No.: US 2002/0006647 A1
(43) Pub. Date: Jan. 17, 2002

(54) Fermentation with a phytase

(75) Inventors: Chris Veit, Wake Forest, NC (US); Claus Felby, Herlev (DK); Larry W. Peckous, Raleigh, NC (US); Hans Sejr Olsen, Holte (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/788,906

(22) Filed: Feb. 20, 2001

Related U.S. Application Data
(60) Provisional application No. 60/185,716, filed on Feb. 23, 2000.

(30) Foreign Application Priority Data
Feb. 23, 2000 (DK) PA 2000 00281

(51) Int. Cl. C12P 7/14

(52) U.S. Cl. 435/162

(57) ABSTRACT

The present invention relates to an improved fermenation process wherein phytic acid-containing material is fermented in the presence of a phytase.

### CROSS-REFERENCE TO RELATED APPLICATIONS

This application claims priority under 35 U.S.C. 119 of Danish application PA 2000 00281 filed Feb. 23, 2000 and U.S. Provisional application Ser. No. 60/185,716, filed Feb. 23, 2000, the contents of which are fully incorporated herein by reference.

### FIELD OF THE INVENTION

The present invention relates to a process of fermenting phytic acid-containing materials; a process of alcohol and other fermented compounds production, in particular ethanol production; the use of phytase activity for saccharification and/or fermentation; and a composition suitable for ethanol production.

### BACKGROUND OF THE INVENTION

Fermentation processes are used for making a vast number of products of big commercial interest. Fermentation is used in industry to produce simple compounds such as alcohols (in particular ethanol); acids, such as citric acid, itaconic acid, lactic acid, gluconic acid; ketones; amino acids, such as glutamic acid, but also more complex compounds such as antibiotics, such as penicillin, tetracyclin; enzymes; vitamins, such as riboflavin, B12, beta-carotene; hormones, which are difficult to produce synthetically. Also in the brewing (beer and wine industry), dairy, leather, tobacco industries fermentation processes are used.

Thus, there is a need for further improvement of fermentation processes and for processes including a fermentation step. Accordingly, the object of the invention is to provide an improved method at least including a fermenting step.

### BRIEF DESCRIPTION OF THE DRAWING

FIG. 1 shows schematically an ethanol production process of the invention.

FIG. 2 shows the results on fermentation of liquefied whole corn mash using AMG and AMG+phytase. CO2 loss vs. time

FIG. 3 shows the phytin level as g/kg dry matter for the corn substrate and following fermentation with AMG and AMG+phytase.

### DETAILED DESCRIPTION OF THE INVENTION

The present invention relates to a process of producing a fermentation product, for instance the ones mentioned in the “Background of the Invention”-section, in particular ethanol, but also beverages, such as beer or wine are contemplated, wherein the fermentation is carried out in the presence of phytase activity. It is to be understood that a carbohydrate source, such as glucose, dextrose, maltose or the like, needs to be present during fermentation for the fermenting organism to be able to ferment. The carbohydrate source may be supplied by direct addition of, e.g., glucose, or may be supplied as a product of, e.g., (pre-)saccharification step, as will be described further below.

Ethanol Production

The process of the invention may in one embodiment be an ethanol process comprising the below steps, wherein phytase activity is added during pre-saccharification and/or fermentation. It is to be understood that the phytase according to the invention may be added during the propagation of yeast cells and/or later on during the actual fermentation. Beverage production, such as beer or wine production is equally contemplated.

Alcohol production, in particular ethanol production, from whole grain can be separated into 4 main steps

Milling

Liquefaction

Saccharification

Fermentation

Milling

In one embodiment the (whole) grain is milled in order to open up the structure and allowing for further processing. Two processes are preferred according to the invention: wet and dry milling. Preferred for ethanol production is dry milling where the whole kernel is milled and used in the remaining part of the process. Wet milling may also be used and gives a good separation of germ and meal (starch granules and protein) and is with a few exceptions applied at locations where there is a parallel production of syrups. Both dry and wet milling is well known in the art of, e.g., ethanol production.

Liquefaction

In an embodiment of the liquefaction step of the invention, milled gelatinized whole grain raw material is broken down (hydrolyzed) into maltodextrins (dextrins) mostly of a DE higher than 4. The hydrolysis may be carried out by acid treatment or enzymatically by alpha-amylase treatment, in particular, with a Bacillus alpha-amylase as will be described further below. Acid hydrolysis is used on a limited basis. The raw material is in one embodiment of the invention milled (whole) grain. However, a side stream from starch processing may also be used.

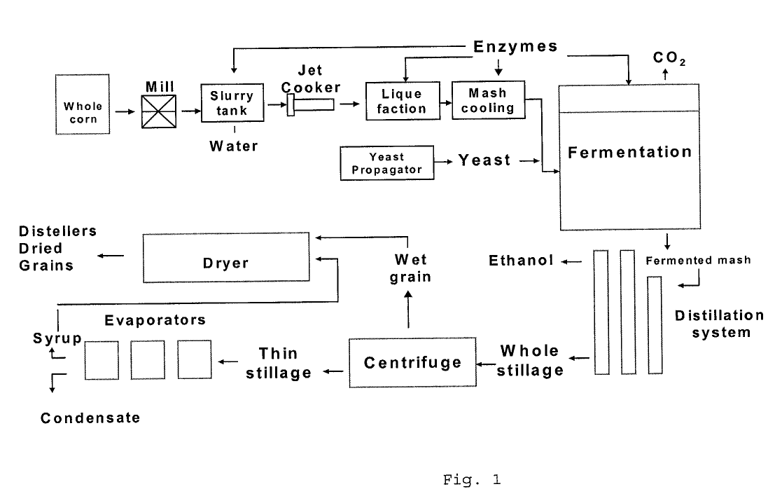

In an embodiment of the invention, enzymatic liquefaction is carried out as a three-step hot slurry process. The slurry is heated to between 60-95° C., preferably 80-85° C. (in the Slurry Tank —see FIG. 1), and the enzyme(s) is(are) added to initiate liquefaction (thinning). Then the slurry is jet-cooked at a temperature between 95-140° C., preferably 105-125° C. to complete gelanitization of the slurry. Then the slurry is cooled to 60-95° C. and more enzyme(s) is(are) added to finalize hydrolysis (secondary liquefaction). The liquefaction process is carried out at pH 4.5-6.5, in particular at a pH between 5 and 6. Milled and liquefied whole grains are known as mash.

Saccharification

To produce low molecular sugars DP₁₋₃ that can be metabolized by yeast, the maltodextrin from the liquefaction must be further hydrolyzed. The hydrolysis is typically done enzymatically by glucoamylases, alternatively, alpha-glucosidases or acid alpha-amylases can be used. A full saccharification step may last up to 72 hours, however, it is common only to do a pre-saccharification of typically 40-90 minutes and then complete saccharification during fermentation (SSF). Saccharification is typically carried out at temperatures from 30-65° C., typically around 60° C., and at pH 4.5.

Fermentation

Yeast typically from Saccharomyces spp. is added to the mash and the fermentation is ongoing for 24-96 hours, such as typically 35-60 hours. The temperature is between 26-34° C., in particular about 32° C., and the pH is from pH 3-6, preferably around pH 4-5.

Note that the most widely used process is a simultaneous saccharification and fermentation (SSF) process where there is no holding stage for the saccharification, meaning that fermenting organism, such as the yeast, and enzyme(s) is(are) added together. When doing SSF it is common to introduce a pre-saccharification step at a temperature above 50° C., just prior to the fermentation.

Distillation

Optionally following the fermentation, the mash may be distilled to extract, for instance, the ethanol.

In the case where the end product is ethanol, obtained according to the process of the invention, it may be used as, e.g., fuel ethanol; drinking ethanol, such as, potable neutral spirits; or industrial ethanol.

By-products

Left over from the fermentation is the grain, which is typically used for animal feed either in liquid or dried form.

Further details on how to carry out liquefaction, saccharification, fermentation, distillation, and recovering of ethanol are well known to the skilled person.

According to the process of the invention the saccharification and fermentation may be carried out simultaneously or separately.

The first aspect the invention relates to a process, wherein phytic acid-containing material is fermented in the presence of a phytase. The addition of a phytase during the fermentation (or, e.g., a combined or simultaneous fermentation and saccharification step, as will be described further below) results in a number of advantages. It is to be understood that the phytase according to the invention may be added during the propagation of yeast cells and/or later on during the actual fermentation. For instance, the addition of phytase results in more free minerals, e.g., Ca²⁺, which are made available to the fermenting organism(s), in particular yeast. Phytic acid interferes with minerals such as especially calcium, magnesium, zinc and iron, in a manner that reduce the stability of a number of alpha-amylases, in particular Bacillus alpha-amylases (e.g., BAN or Bacillus licheniformis alpha-amylases) or Aspergillus alpha-amylases (e.g., the Aspergillus oryzae alpha-amylase sold as FUNGAMYL™ from Novozymes A/S). Therefore, a higher fermentation rate is a result of the hydrolysis of phytin making accessible minerals essential to the yeast metabolism.

In preferred embodiment, the addition of phytase is made in combination with a “Carbohydrate-source generating enzyme”. The term “carbohydrate-source generating enzyme” includes glucoamylases (being a glucose generator), and beta-amylases and maltogenic amylases (being maltose generators).The carbohydrate-source generating enzymes are capable of providing energy to the fermenting microorganism(s) in question.

Further, the availability of more free phosphorus minerals and vitamins—as a result of phytin to inositil conversion—improves the yeast growth and viability during fermentation and thus increases the fermentation and ethanol yields.

Further, the protein availability is increased.

The by-product of an ethanol process may be used as feed (Distillers Dried Grain) and has an improved nutritional value in comparison to a corresponding process where a phytase has not been added.

In one embodiment the invention relates to a process for the production of ethanol, comprising the steps of:

(a) milling whole grains,

(b) liquefying the product of step (a), in the presence of an alpha-amylase,

(c) saccharifying the liquefied material obtained in step (b) in the presence of a phytase,

(d) fermenting the saccharified material obtained in step (c) using a micro-organism; and optionally

(e) distilling of the fermented and saccharified material obtained in step (d).

In a preferred embodiment the whole grains in step a) are dry milled, for instance in a hammer mill.

In an embodiment the DS% (dry solid percentage) in the slurry tank (containing milled whole grains) is in the range from 1-60%, in particular 10-50%, such as 20-40%, such as 25-35%.

In a preferred embodiment of the invention the liquefaction step comprising the following sub-steps:

b1) the hot slurry is heated to between 60-95° C., preferably 80-85° C., and at least an alpha-amylase is added;

b2) the slurry is jet-cooked at a temperature between 95-140° C., preferably 105-125° C. to complete gelanitization of the slurry;

b3) the slurry is cooled to 60-95° C. and more alpha-amylase is added to finalize hydrolysis.

The liquefaction process is in an embodiment carried out at pH 4.5-6.5, in particular at a pH between 5 and 6.

Steps (c) and (d) may be carried out either simultaneously or separately/sequentially. Further, after step (e), an optional ethanol recovery step may be added.

Materials Used in Processes of the Invention

Phytic Acid-containing Material

According to the above described processes of the invention, the raw materials contain phytic acids and fermentable sugars or constituents, which can be converted into sugars. This include starch-containing raw materials, such as tubers, roots, whole grains, corns, cobs, wheat, barley, rye, milo or cereals, sugar-containing raw materials, such as molasses, fruit materials, sugar, cane or sugar beet, potatoes, cellulose-containing materials, such as wood or plant residues.

According to an embodiment of the invention, the phytic acid containing material may be the side stream from starch processing, in particular liquefied starch with a DE of 6-20, in particular between 8-10.

Microorganisms for Fermentation

The microorganism may be a fungal organism, such as yeast or bacteria. Examples of filamentous fungi include strains of Penicillium sp. Preferred organisms for ethanol production is yeasts. Preferred yeast according to the invention is baker's yeast, also known as Saccharomyces cerevisiae. The yeast may according to the invention preferably be added before starting the actual fermentation (i.e., during the propagation phase). The yeast cells may be added in amounts of 10⁵ to 10^{12,} preferably from 10⁷ to 10^{10,} especially 5×10⁷ viable yeast count per ml of fermentation broth. During the ethanol producing phase, the yeast cell count should preferably be in the range from 10⁷ to 10^{10,} more preferably around 2×10^{8.} Example 1 shows a fermentation process of the invention where the yeast is not stressed (yeast count of about 10¹⁰ cells per ml). Even under such conditions, the addition of phytase is shown to improve the fermentation process. Further guidance in respect of using yeast for fermentation can be found in, e.g., “The alcohol Textbook” (Lyons, T. P., Kelsall, D. R; Murtagh, J. E., Publisher: Nottingham University Press, Nottingham, 1995), which is hereby incorporated by reference.

Enzymes

Phytase

The phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any specificity, e.g., be a 3-phytase (EC 3.1.3.8), a 6-phytase (EC 3.1.3.26) or a 5-phytase (no EC number).

In a preferred embodiment the phytase has a temperature optimum in the range from 25-70° C., preferably 28-50° C., more preferably 30-40° C. This is advantageous when the phytase is added during fermentation.

In another preferred embodiment the phytase has a temperature optimum above 50° C., such as in the range from 50-70° C. This is advantageous when the phytase is added during (pre-) saccharification. The dosage of the phytase may be in the range 5.000-250.000 FYT/g DS, preferably 10.000-100.000 FYT/g DS. A preferred suitable dosage of the phytase is in the range from 0.005-25 FYT/g DS, preferably 0.01-10 FYT/g, such as 0.1-1 FYT/g DS.

Here, the phytase activity is determined FYT units, one FYT being the amount of enzyme that liberates 1 micromole inorganic ortho-phosphate per min. under the following conditions: pH 5.5; temperature 37° C.; substrate: sodium phytate (C₆H₆O₂₄P₆Na₁₂) at a concentration of 0.0050 mole/l.

The phytase may be derived from plants or microorganisms, such as bacteria or fungi, e.g., yeast or filamentous fungi.

The plant phytase may be from wheat-bran, maize, soy bean or lily pollen. Suitable plant phytases are described in Thomlinson et al, Biochemistry, 1 (1962), 166-171; Barrientos et al, Plant. Physiol., 106 (1994), 1489-1495; WO 98/05785; WO 98/20139.

A bacterial phytase may be from genus Bacillus, Pseudomonas or Escherichia, preferably the species B. subtilis or E. coli. Suitable bacterial phytases are described in Paver and Jagannathan, 1982, Journal of Bacteriology 151:1102-1108; Cosgrove, 1970, Australian Journal of Biological Sciences 23:1207-1220; Greiner et al, Arch. Biochem. Biophys., 303, 107-113, 1993; WO 98/06856; WO 97/33976; WO 97/48812.

A yeast phytase or myo-inositol monophosphatase may be derived from genus Saccharomyces or Schwanniomyces, preferably species Saccharomyces cerevisiae or Schwanniomyces occidentalis. The former enzyme has been described as a Suitable yeast phytases are described in Nayini et al, 1984, Lebensmittel Wissenschaft und Technologie 17:24-26; Wodzinski et al, Adv. Appl. Microbiol., 42, 263-303; AU-A-24840/95;

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trametes, preferably the species Aspergillus terreus, Aspergillus niger, Aspergillus niger var. awamori, Aspergillus ficuum, Aspergillus fumigatus, Aspergillus oryzae, T. lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322:1275-1282; Piddington et al., 1993, Gene 133:55-62; EP 684,313; EP 0 420 358; EP 0 684 313; WO 98/28408; WO 98/28409; JP 7-67635; WO 98/44125; WO 97/38096; WO 98/13480.

Modified phytases or phytase variants are obtainable by methods known in the art, in particular by the methods disclosed in EP 897010; EP 897985; WO 99/49022; WO 99/48330.

Commercially available phytases contemplated according to the invention include BIO-FEED PHYTASE™, PHYTASE NOVO™ CT or L (all from Novozymes), or NATUPHOS™ NG 5000 (from DSM).

Alpha-amylase

The liquefaction step may be performed in the presence of an alpha-amylase derived from a microorganism or a plant. Preferred alpha-amylases are of fungal or bacterial origin. Bacillus alpha-amylases (often referred to as “Termamyl-like alpha-amylases”), variant and hybrids thereof, are specifically contemplated according to the invention. Well-known Termamyl-like alpha-amylases include alpha-amylase derived from a strain of B. licheniformis (commercially available as Termamyl™) B. amyloliquefaciens, and B. stearothermophilus alpha-amylase (BSG). Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. In the context of the present invention, a Termamyl-like alpha-amylase is an alpha-amylase as defined in WO 99/19467 on page 3, line 18 to page 6, line 27. Contemplated variants and hybrids are described in WO 96/23874, WO 97/41213, and WO 99/19467. Contemplated alpha-amylase derived from a strain of Aspergillus includes Aspergillus oryzae and Aspergillus niger alpha-amylases. Commercial alpha-amylase products and products containing alpha-amylases include TERMAMYL™ SC, FUNGAMYL™, LIQUOZYME™ and SAN™ SUPER.

Fungal alpha-amylases may be added in an amount of 0.001-1.0 AFAU/g DS, preferably from 0.002-0.5 AFAU/g DS, preferably 0.02-0.1 AFAU/g DS.

Bacillus alpha-amylases may be added in effective amounts well known to the person skilled in the art.

Glucoamylase

The saccharification step (c) or a combined saccharification and fermentation step (SSF step) may be carried out in the presence of a glucoamylase derived from a microorganism or a plant. Preferred is glucoamylase of fungal or bacterial origin selected from the group consisting of Aspergillus glucoamylases, preferably, A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other suitable Aspergillus glucoamylase variants include variants to enhance the thermal stability: G137A and G139A (Chen et al. (1996), Prot. Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Furthermore Clark Ford presented a paper on Oct 17, 1997, ENZYME ENGINEERING 14, Beijing/China Oct 12-17, 97, Abstract number: Abstract book p. 0-61. The abstract suggests mutations in positions G137A, N20C/A27C, and S30P in an Aspergillus awamori glucoamylase to improve the thermal stability.

Other glucoamylases include Talaromyces glucoamylases, preferably derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases preferably include glucoamylases from the genus Clostridium, more preferably, C. thermoamylolyticum (EP 135,138), and C. thermohydrosulfuricum (WO 86/01831).

Glucoamylases may in an embodiment be added in an amount of 0.02-2 AGU/g DS, preferably 0.1-1 AGU/g DS, such as 0.2 AGU/g DS

The ratio between acidic fungal alpha-amylase activity (AFAU) per glucoamylase activity (AGU) (AFAU per AGU) may in one embodiment be at least 0.1, in particular at least 0.16, such as in the range from 0.12 to 0.30.

Commercial products include SAN™ SUPER and AMG™ E (from Novozymes); OPTIDEX™ 300 (from Genencor Int.); AMIGASE™ (from DSM); G-ZYME™ G900 (from Enzyme Bio-Systems).

Protease

Addition of protease(s) increase(s) the FAN (Free amino nitrogen) level and increase the rate of metabolism of the yeast and further gives higher fermentation efficiency.

Suitable proteases include fungal and bacterial proteases. Preferred proteases are acidic proteases, i.e., proteases characterized by the ability to hydrolyze proteins under acidic conditions below pH 7.

Suitable acid fungal proteases include fungal proteases derived from Aspergillus, Mucor, Rhizopus, Candida, Coriolus, Endothia, Enthomophtra, Irpex, Penicillium, Sclerotium and Torulopsis. More preferred are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae; and acidic proteases from Mucor pusillus or Mucor miehei.

Bacterial proteases, which are not acidic proteases, include the commercially available products Alcalase® and Neutrase® (available from Novozymes A/S).

Protease(s) may in one embodiment be added in an amount of 10⁻⁷ to 10⁻⁵ gram active protease protein/g DS, in particular 10⁻⁷ to 5×10⁻⁶ gram active protease protein/g DS

Additional Enzyme

One or more additional enzymes may also be incorporated. Additional enzymes include pullulanases.

Another aspect the invention relates to the use of phytase, in particular the phytases mentioned above for saccharification and/or fermentation (SSF) and for use for ethanol production.

This aspect of the invention relates to a composition comprising a phytase and at least one carbohydrate-source generating enzyme (as defined above), preferably, a glucoamylase, such as an Aspergillus niger and/or Talaromuces emersonii glucoamylase. The composition may further comprise a protease, preferably, an acid protease, such as an acid fungal protease.

Materials and Methods

Materials:

Phytase: derived from Peniophora lycii (LN 0 1305 dept. 550) E-1999-0082) (available from Novozymes)

Glucoamylase: A. niger glucoamylase (available as AMG E (1999-SE-0025) from Novozymes)

Alpha-amylase: BSG (B. stearothermophilus alpha-amylase which is available from Novozymes as TERMAMYL™ SC)

Mash: Liquefied whole corn mash prepared by a hot slurry process and Termamyl SC. The mash had a DE of about 17 and a dry substance of 28%.

Yeast: Dry yeast (Saccharomyces cervisiae)

Determination of Alpha-Amylase Activity (KNU)

1. Phadebas Assay

Alpha-amylase activity is determined by a method employing Phadebas® tablets as substrate. Phadebas tablets (Phadebas® Amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temp., pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

2. Alternative Method

Alpha-amylase activity is determined by a method employing the PNP-G7 substrate. PNP-G7 which is a abbreviation for p-nitrophenyl-alpha,D-maltoheptaoside is a blocked oligosaccharide which can be cleaved by an endo-amylase. Following the cleavage, the alpha-Glucosidase included in the kit digest the substrate to liberate a free PNP molecule which has a yellow colour and thus can be measured by visible spectophometry at λ=405 nm. (400-420 nm.). Kits containing PNP-G7 substrate and alpha-Glucosidase is manufactured by Boehringer-Mannheim (cat. No. 1054635).

To prepare the substrate one bottle of substrate (BM 1442309) is added to 5 ml buffer (BM1442309). To prepare the alpha-Glucosidase one bottle of alpha-Glucosidase (BM 1462309) is added to 45 ml buffer (BM1442309). The working solution is made by mixing 5 ml alpha-Glucosidase solution with 0.5 ml substrate.

The assay is performed by transforming 20 micro l enzyme solution to a 96 well microtitre plate and incubating at 25° C. 200 micro l working solution, 25° C. is added. The solution is mixed and pre-incubated 1 minute and absorption is measured every 15 sec. over 3 minutes at OD 405 nm.

The slope of the time dependent absorption-curve is directly proportional to the specific activity (activity per mg enzyme) of the alpha-amylase in question under the given set of conditions.

Determination of Acid Amylolytic Activity (FAU)

One Fungal Alpha-Amylase Unit (1 FAU) is defined as the amount of enzyme, which breaks down 5.26 g starch (Merck Amylum solubile Erg. B.6, Batch 9947275) per hour at Novo Nordisk's standard method for determination of alpha-amylase based upon the following standard conditions:

| | |
|---|---|
| Substrate | Soluble starch |
| Temperature | 37° C. |
| pH | 4.7 |
| Reaction time | 7-20 minutes |

A detailed description of Novo Nordisk's method is available on request.

Determination of Acid Alpha-amylase Activity (AFAU)

Acid alpha-amylase activity is measured in AFAU (Acid Fungal Alpha-amylase Units), which are determined relative to an enzyme standard.

The standard used is AMG 300 L (wild type A. niger G1 AMG sold by Novo Nordisk). The neutral alpha-amylase in this AMG falls after storage at room temperature for 3 weeks from approx. 1 FAU/mL to below 0.05 FAU/mL.

The acid alpha-amylase activity in this AMG standard is determined in accordance with AF 9 ⅓ (available from Novo method for the determination of fungal alpha-amylase). In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

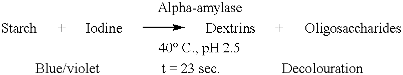

Standard conditions/reaction conditions: (per minute)

| | |
|---|---|
| Substrate | starch, approx. 0.17 g/L |
| Buffer | Citrate, approx. 0.03 M |
| Iodine (I₂) | 0.03 g/L |
| CaCl₂ | 1.85 mM |
| pH | 2.50 ± 0.05 |
| Incubation temperature | 40° C. |
| Reaction time | 23 seconds |
| Wavelength | Lambda = 590 nm |
| Enzyme concentration | 0.025 AFAU/mL |
| Enzyme working range | 0.01-0.04 AFAU/mL |

Further details can be found in EB-SM-0259.02/01 available on request from Novozymes, and hereby incorporated by reference.

Determination Of Glucoamylase Activity (AGU)

The Novo Amyloglucosidase Unit (AGU) is defined as the amount of enzyme, which hydrolyzes 1 micromole maltose per minute at 37° C. and pH 4.3. A detailed description of the analytical method (AEL-SM-0131) is available on request from Novo Nordisk.

The activity is determined as AGU/ml by a method modified after (AEL-SM-0131, available on request from Novo Nordisk) using the Glucose GOD-Perid kit from Boehringer Mannheim, 124036. Standard: AMG-standard, batch 7-1195, 195 AGU/ml. 375 microL substrate (1% maltose in 50 mM Sodium acetate, pH 4.3) is incubated 5 minutes at 37?C. 25 microL enzyme diluted in sodium acetate is added. The reaction is stopped after 10 minutes by adding 100 microL 0.25 M NaOH. 20 microL is transferred to a 96 well microtitre plate and 200 microL GOD-Perid solution (124036, Boehringer Mannheim) is added. After 30 minutes at room temperature, the absorbance is measured at 650 nm and the activity calculated in AGU/ml from the AMG-standard.

### EXAMPLES

### Example 1

Fermentation in the Presence of Phytase

250 g of mash was filled into a 500 mL blue cap bottle. The pH of the mash was adjusted to 4.5. Before fermentation a pre-saccharification step was carried out by adding the saccharification enzymes and placing the bottles in a water bath at 60° C. for 70 minutes. The bottle was cooled in a water bath for 40 minutes to 30° C. and dry yeast was added at a dosage of 0.8 g/bottle (in order to reach 30° C. within 40 minutes ice is added to the water bath). The bottle is closed by a yeast-lock filled with concentrated H₂SO₄. The fermentation was continued for 96 hours and the fermentation rate was monitored by weighing the bottle at regular intervals for measuring CO₂ loss.

Determination of Phytin Content

The phytin content was measured for all treatments and the substrate. The phytin content was measured by The Danish Institute of Agricultural sciences, Tjele, Denmark, according to the method described in: Brooks, J. R. and C. V. Morr. 1984. Phosphorus and phytate content of soybean protein components. J. Agric. Food Chem. 32: 872-874.

Treatments

Three different treatments were applied (all enzyme dosages are based on dry matter):

0.2 AGU/g AMG E

0.2 AGU/g AMG E+1.0 FYT/g phytase

1.0 FYT/g phytase

Each treatment was duplicated 4 times.

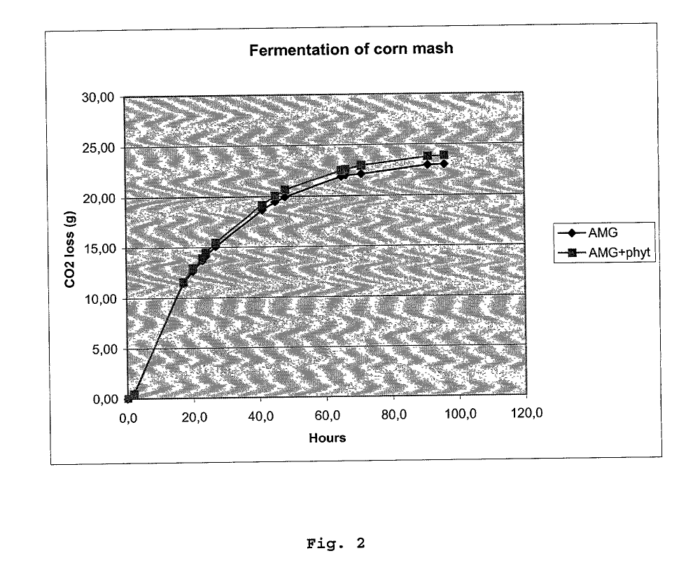

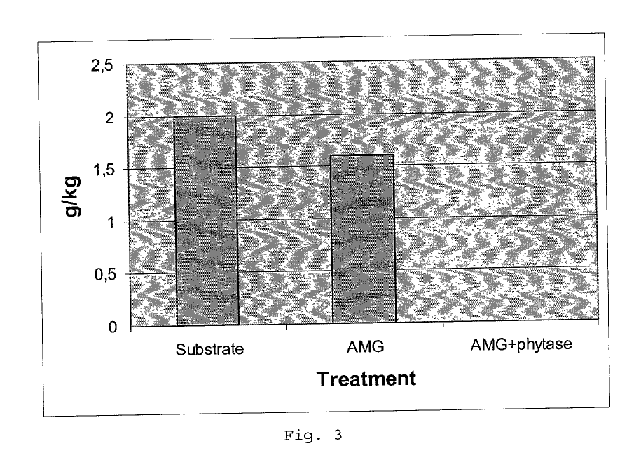

The results of the fermentations are shown in FIG. 2 and Table 1. The positive effect of the phytase is significant at a level of P=0.05. The effect of the phytase treatment on the level of phytin is shown in FIG. 3. The phytase efficiently hydrolyses the phytin below the detection level.

TABLE 1
Fermentation of whole corn mash. CO₂ loss after
96 hour fermentation using AMG E and phytase.

| | CO₂ (g) | | |
|---|---|---|---|
| | | 0.2 AGU/g + | |
| Hours | 0.2 AGU/g | 1 FYT/g | 1 FYT/g |
| CO₂ loss 96 hr. | 23.0 | 23.9 | 5.3 |
| % of theoreti- | 88.0 | 91.1 | 20.2 |
| cal yield at 96 | | | |
| hours | | | |

1. A process of fermenting a phytic acid-containing material, comprising fermenting said phytic acid-containing material in the presence of a phytase.

2. The process of claim 1, wherein said phytase is derived from a strain of Peniophra lycii, Aspergillus oryzae, Aspergillus ficumm, or Aspergillus niger.

3. The process of claim 1, wherein said phytase has a temperature optimum in the range of 20-70° C.

4. The process of claim 1, wherein said phytase has a temperature optimum of above 50° C.

5. The process of claims 1, wherein said phytic acid-containing material is fermented with a micro-organism capable of fermenting sugars or converted sugars.

6. The process of claim 5, wherein said micro-organism is a yeast.

7. The process of claim 5, wherein said micro-organism is Saccharomyces cerevisae.

8. The process of claim 1, wherein said phytic acid-containing material is derived from whole grains or a side stream from starch processing.

9. The process of claim 1, wherein said phytic acid-containing material is derived from liquefied starch with a DE of 8-10.

10. The process of claim 1, wherein said fermentation is carried out in the presence of a carbohydrate-generating enzyme.

11. The process of claim 10, wherein said carbohydrate-generating enzyme is a glucoamylase.

12. The process of claim 1, wherein said fermentation is carried out in the presence of a protease.

13. The process of claim 12, wherein said protease is a fungal protease.

14. The process of claim 12, wherein said protease is derived from a strain of Aspergillus.

15. The process of claim 12, wherein said protease is derived from Aspergillus niger.

16. The process of claim 12, wherein said protease is a bacterial protease.

17. The process of claim 12, wherein said protease is an acidic, neutral or alkaline protease.

18. The process of claim 12, wherein said protease is derived from a strain of Bacillus.

19. The process of claim 1, wherein the product of said fermentation is ethanol.

20. A process for the production of ethanol, comprising the steps of:
(a) milling whole grains,
(b) liquefying the product of said (a)in the presence of an alpha-amylase,
(c) saccharifying the liquefied material obtained in said (b), and
(d) fermenting the saccharified material obtained in said (c) using a micro-organism.

21. The process of claim 20, further comprising (e) distilling the fermented and saccharified material obtained in step (d).

22. The process of claim 20, further comprising adding a carbohydrate-source generating enzyme in said (c).

23. The process of claim 22, wherein said carbohydrate-source generating enzyme is a glucoamylase.

24. The process of claim 20, wherein said (b) comprises:
i) heating a hot slurry comprising milled whole grains from said (a) to between 60-95° C. and adding at least one alpha-amylase to said slurry;
ii) jet-cooking said slurry at a temperature between 95-140° C. to complete gelanitization of said slurry; and
iii) cooling said slurry to 60-95° C. and adding alpha-amylase to complete hydrolysis.

25. The process of claim 20, wherein said (b) is carried out at pH of 4.5-6.5.

26. The process of claim 20, wherein said (b)is carried out at a pH between 5 and 6.

27. The process of claim 20, wherein a phytase is added during said (c) or prior to said (c) (pre-saccharification).

28. The process of claim 20, wherein a phytase is added during said (c) and/or said (d).

29. The process of claim 20, wherein a phytase is added during said (c).

30. The process of claim 20, wherein a phytase is added during said (d).

31. The process of claim 20, wherein said (c) and said (d) are carried out simultaneously.

32. The process of claim 20, wherein said (c) and said (d) are carried out sequentially.

33. The process of claim 21, further comprising recovering the ethanol after said (e).

34. The process of claim 20, wherein said (a) is a dry milling step or wet milling step.

35. The process of claim 20, wherein said alpha-amylase is derived from a strain of the genus Bacillus or a strain of Aspergillus.

36. The process of claim 28, wherein said phytase is derived from a strain of Peniophra lycii or Aspergillus oryzae.

37. The process of claims 20, wherein said (d) is carried out using a micro-organism capable of fermenting sugars to ethanol.

38. The process of claim 37, wherein said micro-organism is a yeast.

39. The process of claim 37 wherein said micro-organism is Saccharomyces cerevisae.

40. The process of claim 20, wherein said (d) is carried out in the presence of carbohydrate-source generating enzyme.

41. The process of claim 40, wherein said carbohydrate-source generating enzyme is a glucoamylase.

42. The process of claim 20, wherein said (d) is carried out in the presence of a protease.

43. The process of claim 42, wherein said protease is a fungal protease.

44. The process of claim 42, wherein said protease is an acid fungal protease derived from a strain of Aspergillus.

45. The process of claim 42, wherein said protease is an acid fungal protease derived from a strain of Aspergillus niger.

46. The process of claim 42, wherein said protease is an acid, neutral or alkaline protease.

47. The process of claim 42, wherein said protease is derived from a strain of Bacillus.

48. A process for the production of ethanol, comprising the steps of:
(a) providing a phytic acid-containing material,
(b) liquefying said phytic acid-containing material in the presence of an alpha-amylase,
(c) saccharifying the liquefied material obtained in said (b), and
(d) fermenting the saccharified material obtained in said (c) using a micro-organism.

49. The process of claim 48, wherein the phytic acid-containing material is milled whole grain or a side stream from starch processing.

50. A composition comprising at least one phytase and at least one carbohydrate-generating enzyme.

51. The composition of claim 50, wherein said at least one carbohydrate-generating enzyme is a glucoamylase.

52. The composition of claim 51, further comprising a protease.

53. The composition of claim 52, wherein said protease is an acid protease.

54. The composition of claim 52, wherein said protease is an acid fungal protease.
